(19) United States
(12) Patent Application Publication
(10) Pub. No.: US 2024/0358399 A1
(43) Pub. Date: Oct. 31, 2024

(54) SYSTEM AND METHOD FOR IMAGE GUIDED INTERVENTIONS

(71) Applicant: CLEAR GUIDE MEDICAL, INC., Baltimore, MD (US)

(72) Inventors: Pezhman Foroughi, Baltimore, MD (US); Paul C. Clark, Bethesda, MD (US); Martin Hossbach, Baar-Ebenhausen (DE); Purnima Rajan, Baltimore, MD (US)

(21) Appl. No.: 18/553,404

(22) PCT Filed: Mar. 31, 2022
(86) PCT No.: PCT/US2022/022960

§ 371 Date: Sep. 29, 2023

Related U.S. Application Data
(60) Provisional application No. 63/168,911, filed on Mar. 31, 2021.

(51) Int. Cl. A61B 17/34; A61B 90/00

(52) U.S. Cl. CPC A61B 17/3403; A61B 90/36; A61B 90/39; A61B 2090/366; A61B 2090/371; A61B 2090/372; A61B 2090/374; A61B 2090/3995

(57) ABSTRACT

A guidance system for targeted interventions using medical instruments may include an optical unit positioned such that a desired area of intervention is within a field of view. A control unit having a touch screen display, may be communicatively coupled with the optical unit and configured to receive a real time image of the area of intervention, and further configured to (i) receive MRI volume representations of the desired area of intervention, (ii) register any multi modal markers that appear in the field of view, (iii) generate guidance feedback images comprising a plurality of image elements based on the MRI volume representations and (iii) to transmit the guidance feedback images to the projector. The projector in turn directs at least one of the guidance feedback image elements onto the area of intervention responsive to receipt of the guidance feedback images from said control unit.

### BACKGROUND

### 1. Technical Field

The present disclosure generally relates to image systems and methods for image guided interventions.

### 2. Discussion of Related Art

The use of advanced imaging for diagnosis and to guide interventions continues to grow. From recent market reports, the field of image-guided surgical procedures is a $3 billion market worldwide expected to grow to $5 billion by 2023. The interventional MRI market is estimated to be 10% of the number of in-house MRI scans or 1.7 million procedures per year in the US. With the number of MRI machines growing 10% annually, the number of MR-guided interventions is expected to grow as well.

Specifically, CT and MRI provide three-dimensional cross-sectional imaging and are increasingly used in image-guided interventions, which can provide minimally-invasive treatment options for patients. However, there is increasing concern about the use of CT imaging and associated ionizing radiation, with over 57 million in-hospital scans performed annually in the United States. As radiation exposure concerns continue to rise for both physicians [Roguin-2013] and patients, the use of MRI is increasing, with 18 million in-hospital scans performed annually.

Interventional MRI combines multiplanar cross-sectional imaging capabilities with exquisite soft tissue contrast. MRI guidance has been used for percutaneous needle injections to diagnose and treat neuropathic pain, perform needle biopsy, drainage, tumor ablation, and other clinical indications. The high tissue contrast of MRI makes interventional MRI optimal for lesions that may be difficult to visualize with other modalities such as ultrasound, fluoroscopy and CT. As currently performed, MRI-guided interventions use an “advance and check” method. This can be challenging, consisting of several steps: 1) initial MRI scan to visualize the anatomy of interest; 2) path planning based on these images; 3) moving the patient out of the scanner bore and manual placement of the needle; 4) moving the patient back into the scanner bore and verification of needle placement with repeat imaging; and finally, 5) taking a sample or injecting a drug. The needle placement and verification steps typically require multiple attempts using the advance and check method and rely on the surgeon's spatial skills to make needle orientation adjustments based on the new images (“cognitive fusion”). These steps take time, especially moving the patient in and out of the scanner bore and re-imaging with each needle advance, while the patient must remain still inside the scanner, thereby increasing patient discomfort (and anesthesia duration if used). Such multi-step MRI-guided procedures increase the MRI room time and overall procedure cost and can also cause logistical problems in scanner usage and physician scheduling. Consequently, most interventional procedures in adults are still performed using CT, fluoroscopy, and ultrasound, which do not provide the same image quality as MRI but can be quickly obtained.

### SUMMARY OF THE DISCLOSURE

Aspects of the invention may involve systems and methods. In one embodiment, a guidance system for targeted interventions may be provided. The system may include an optical unit having first and second cameras and a projector disposed between the first and second cameras. The optical unit may be positioned such that a desired area of intervention on a subject is within its field of view. The are of intervention may be defined by plurality of multi modal markers. A may be communicatively coupled with the optical unit and include a touch screen display. The control unit may be configured to receive a real time image of the area of intervention from the optical unit. The control unit may be further configured to (i) receive MRI volume representations of the desired area of intervention, (ii) register multi modal markers that appear in the field of view, (iii) generate guidance feedback images comprising a plurality of image elements based on the MRI volume representations and the real time image responsive to multi modal marker registration and (iii) to transmit the guidance feedback images to the projector. The projector may then direct at least one of the guidance feedback image elements onto the area of intervention responsive to receipt of the guidance feedback images from the control unit.

In another embodiment, a method of guiding an instrument to a target for guided intervention may be provided. In this embodiment, a volumetric image of an intervention area may be displayed on a touch screen display. An instrument path may be generated such that it overlays the volumetric image in responsive to input from a user contacting the touch screen display at selected points of the volumetric image. A guidance feedback image may be generated responsive to instrument contact with the intervention area of the subject, where the guidance feedback image includes a plurality of image elements. A first image element may be projected onto the subject where the first image element indicates any entry point for a medical instrument. A second image element may be projected onto the subject where the second image element indicates a trajectory guide for the medical instrument. The second image element may be a dynamic shadow line that contracts to a point when the instrument is aligned with the instrument path. A third image element may be projected onto the subject where the third image element indicates a distance from a tip of the medical instrument to the target.

### BRIEF DESCRIPTION OF THE DRAWINGS

FIG. 1 shows components of a guidance system according to an embodiment of the invention.

FIG. 2 shows a guidance system for interventions according to an embodiment of the invention.

FIG. 3 shows an optical head of the guidance system of FIG. 1.

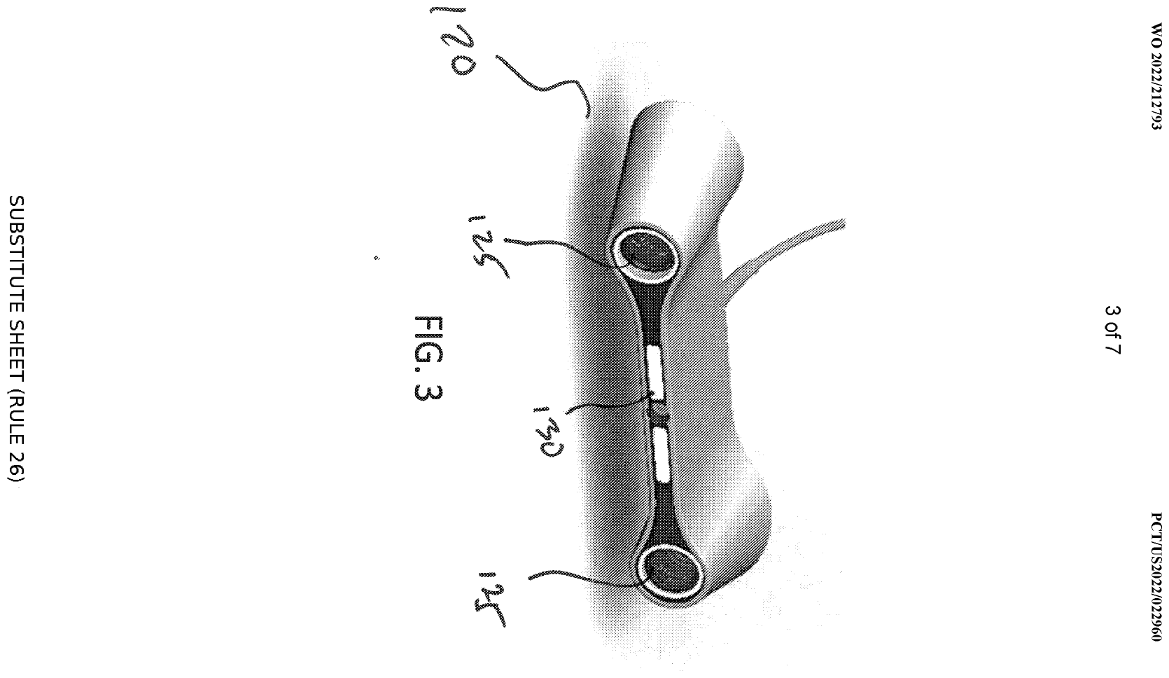

FIG. 4 depicts a guidance system positioned proximate to an MRI bore including touch screen displaying real time and volumetric images.

FIG. 5 illustrates the guidance system of FIG. 4 illustrating a front view of a display screen.

FIG. 6 shows a needle in a first position in contact with a phantom with guidance image elements are projected onto the phantom.

FIG. 7 depicts a needle in a second position in contact with a phantom with guidance image elements are projected onto the phantom.

### DETAILED DESCRIPTION

The present disclosure describes medical-device systems and methods that allow operators to perform targeted and non-targeted navigation-assisted interventions using certain types of intervention instruments, such as needle-like instruments in some cases using visual or automatic termination criteria. Example applications may include needle biopsies, tumor ablations, catheter insertion, orthopedic interventions, and other instruments, all of which may use several types of image data. Typically, these instruments are inserted into a patient's body at very specific locations, orientations, and depths to reach predetermined target areas, where they perform an instrument-specific action or function, which may include tissue sampling, heating, cooling, liquid deposition, suction, or serving as a channel for other objects.

Clear Guide Medical has previously developed a novel visual tracking technology platform based on real-time camera-based computer vision, and embodied the tracking platform in products for ultrasound-based systems and computerized tomography (CT)-based systems for image/instrument guidance and multi-modality fusion. Certain aspects of this technology have already been described in U.S. patent application Ser. Nos. 13/648,245, 14/092,755, 14/092,843, 14/508,223, 14/524,468, 14/524,570, and 14/689,849 (collectively referred to as “Clear Guide's Patent Applications”).

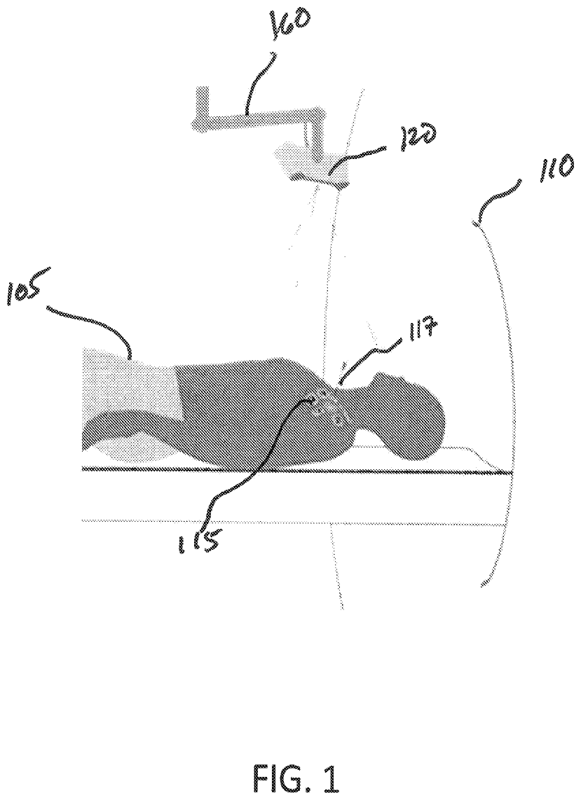

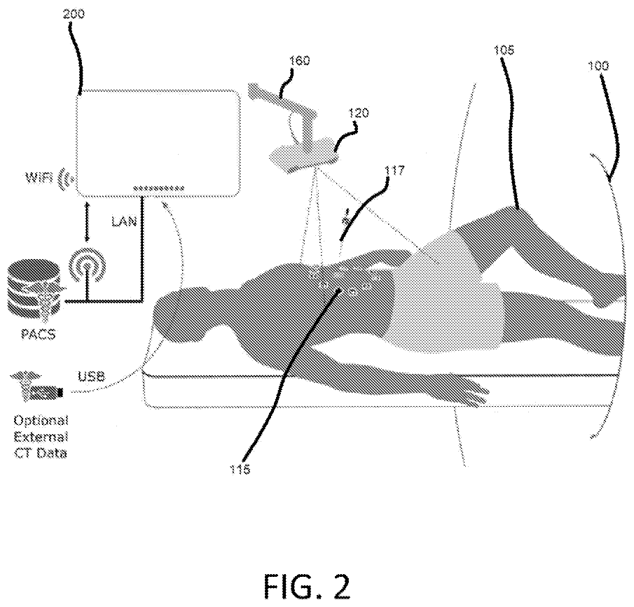

The present invention provides a system and method to assist clinicians/users in accurate and fast image-guided instrument placement when using volumetric imaging without ultrasound which is suitable for use inside the MR suite or, in some embodiments, in bore. FIG. 1 depicts an embodiment of a system according to the present invention in use with an MR device while FIG. 2 depicts an embodiment of a system in use with a CT device. A patient 105 is positioned on an imaging device gantry 112 next to the imaging device 110, i.e., an MR device in FIG. 1 and a CT scanner in FIG. 12. The patient has a plurality of multi-modal markers 115 applied in an irregular fashion around an intervention area and a medical instrument 117 is shown slightly above the intervention area. An optical unit 120 is communicatively coupled with a control unit 200. Control unit 200 transmits dynamic, real time guidance feedback to optical unit 120 which projects a real-time interactive visual guidance and tracking indicators.

The optical unit 120 may be positioned above the patient similar to standard OR lighting and adjusted so that the area of intervention is within its field of view, similar to standard OR lighting. Note that the projector can facilitate correct orientation of the optical unit by highlighting the viewing area.

Optical unit 120 may include one or more cameras 125 and a projector 130 disposed within a housing 135. In some embodiments, optical unit 120 may be MR conditional. By MR conditional it is meant that optical unit 120 exhibits little to no magnetic attraction towards the MRI bore even at close proximities and it is fully operational at the 300 Gauss line.

In at least one embodiment, as shown in FIG. 2 projector 130 is disposed between and rigidly attached to first and second cameras 125 within housing 135 such that the relative position of cameras 125 and projector 130 is fixed. In some embodiments, the cameras 125 may be attached to projector 130 via a stabilizer bar. Other known mechanisms for rigid attachment may also be used.

Cameras 125 may be any suitable camera that provides full color images with a resolution of 1080p or higher, low noise levels and a frame rate of at least 30 fps. In some embodiments, camera 125 may comprise board level cameras.

Projector 130 should be capable of displaying dynamic visual guidance and tracking indicators onto the body of a patient that are readily visible with the unaided human eye. To that end, projector 130 may have a small footprint, exhibit low power consumption and have a brightness of 1501lm or greater. In addition, projector 130 may have a low latency, i.e., 100 ms or less. Suitable projectors 130 may be board level projector modules including laser projector modules and digital light processing (DLP) modules.

In some embodiments, USB 3.0 technology may be used to both deliver power to projector 130 and send a video feed as well. In such embodiments, an interface electronics board (not shown) may be provided to receive images and forward those images to projector 130. The interface electronics board may comprise, for example, a Cypress CX3 USB controller and one or more small ARM-based processors. The interface electronics board may be configured to interface with industry standard single-board-computers, camera modules and optics engines.

Control unit 200 is communicatively coupled with optical unit 120, via, for example, a USB 3.0 connection, and may be configured to receive a real time image of a desired area of intervention of the patient. Control unit 200 is further configured to receive volumetric images such as MRI images or CT scans, through the PACS system or from an external storage device, e.g., USB stick, an external hard drive or some other volumetric image storage medium.

Control unit 200 is further configured to segment the multi-modal markers in the image volume and register the multi modal markers when the patient is brought into the field of view of cameras 125. Control unit 200 is further configured to receive images from cameras 125, in some embodiments, high definition, stereo images, and send feedback signals to projector 130.

Control unit 200 may be a tablet-based medical grade PC, e.g., a Tangent T13 Medical Tablet PC available from Tangent, Inc. of Burlingame, California. In some embodiments. To facilitate MR compatibility, any structural ferromagnetic elements may be removed and replaced with non-ferromagnetic substitutes thereby rendering Control unit 200 MRI safe.

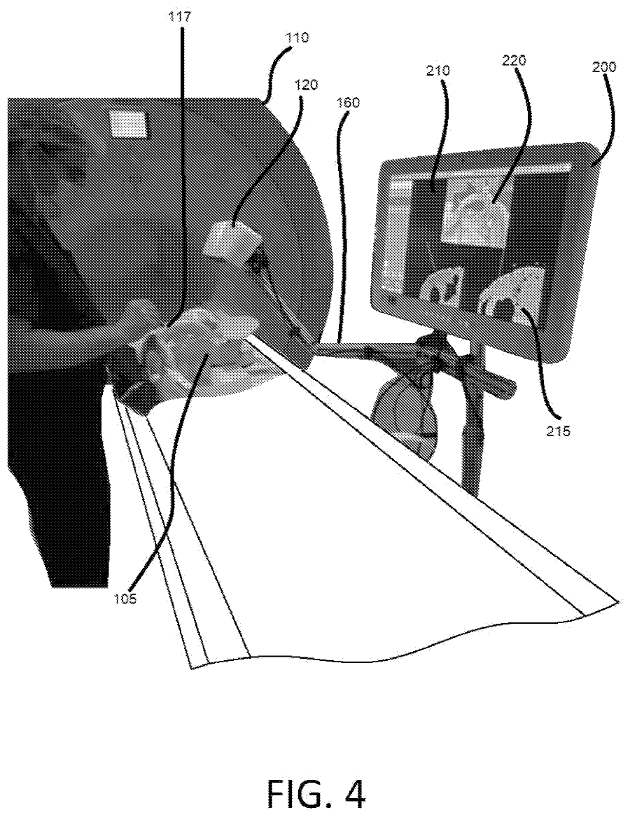

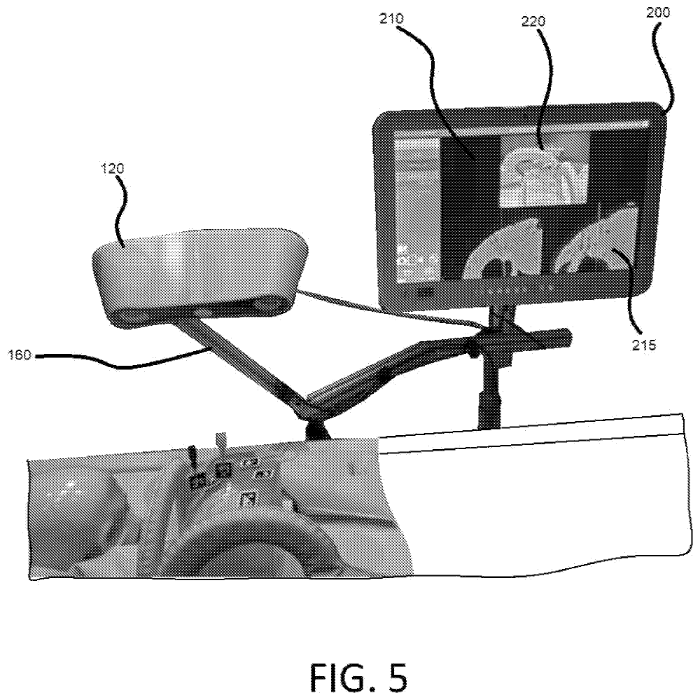

As shown in FIGS. 4 and 5, control unit 200 may also be provided with one or more touch screen displays 210 that are configured to display volumetric images 215 generated by imaging device 110 and images of the patient 220 captured by cameras 110.

In keeping with an aspect of the invention, optical unit 120 may be positioned such that the area of intervention is within the field of view of cameras 125. To that end optical unit 120 may be attached to a mounting arm 160. In turn, mounting arm 160 may be attached to a wall or ceiling of the operating room or to a pole cart 165 which may hold control unit 200 and/or other instruments. Optical unit 120 may be fixedly attached to mounting arm 160 or removably attached thereto. In some embodiments, optical unit 120 may be rotatably mounted to mounting arm 160 in a manner that allows optical unit 120 to rotate about a mounting point of mounting arm 160 with up to 360° of freedom. In some embodiments, optical unit 120 may be attached to mounting arm 160 via a ball joint (not shown).

Mounting arm 160 may be comprised of a non-ferromagnetic material and may include a single arm segment or may include two or more arm segments articulatably connected to one another. Mounting arm 160 may include a handle at the distal end to allow repositioning. Accordingly, optical unit 120 may be adjusted so that cameras 125 can capture any desired target in their field of view. The clinician/user may adjust the positioning of optical unit 120 by viewing the intervention area and changing the orientation of the optical unit 120 until image 220 displays the desired target.

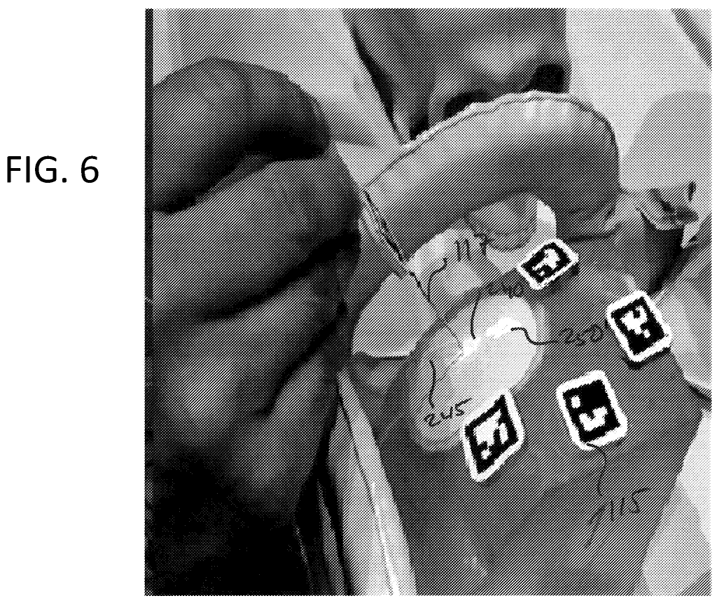

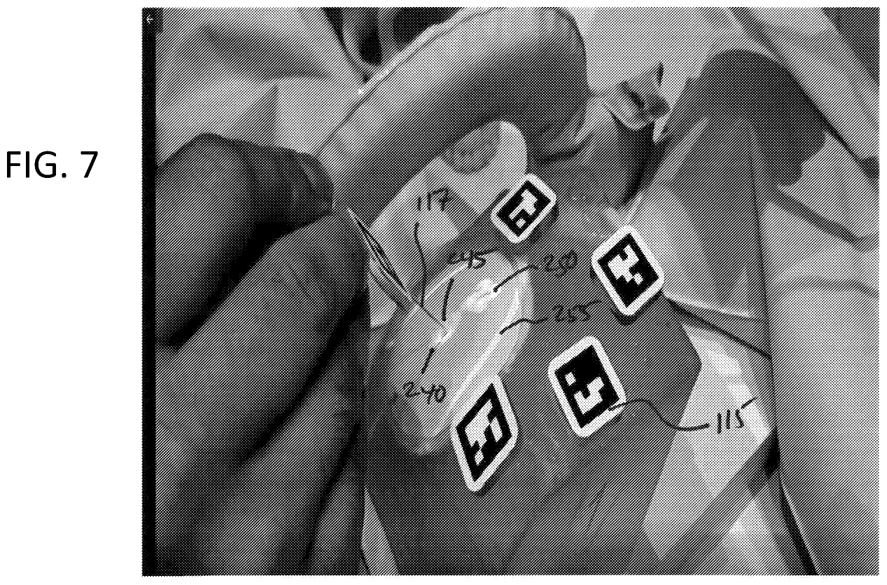

In operation, after images are registered, the clinician/user may interact with control unit 200 and select an entry point and a target to define a planned intervention path for instrument 117. In at least one embodiment, the volumetric image of the area may be displayed on the touch screen. The user/clinician may contact the desired target and entry point to create a trajectory path for the instrument. The clinician/use may then contact with the area of intervention with instrument 117, control unit 200 will generate a guidance feedback image which may include multiple image elements. In keeping with the invention, as illustrated in FIGS. 6 and 7, the guidance feedback image may include at least one of a first mage element 240 representing a point of entry of medical instrument 117, a second image element 245 representing an orientation of medical instrument 117 and third and fourth image elements 250, 255 both representing the distance from the tip of medical instrument 117 to a target. In some embodiments, the first image element 240 is a closed geometric shape such as a circle. In some embodiments, the second image element 245 is a straight line. In some embodiments, the third image element 250 is an alphanumeric character and the fourth image element 255 is a closed, dynamic geometric shape.

Projector 130 may project the guidance feedback image onto the body of the patient responsive to marker registration. The first image element 240 may be projected on the patient's body to denote an entry point where, for example, the tip of instrument 117 should be placed. The first image element 240 may be a small circle of a color that is readily visible to the unaided human eye. In some embodiments the first image element 240 is red.

Once instrument 117 is sufficiently close to the entry point, the second image element 245 may be projected onto the patient's body to guide the orientation of instrument 117 as it is advanced. The second image element 245 may be a line which acts as a virtual needle shadow to guide orientation of instrument 117. As the orientation of instrument 117 changes, the shadow line extends and/or contracts. When the shadow line is minimized, instrument 117 is oriented such that it will pass very close to or hit the target when advanced in that orientation. In some embodiments, the second image element 245 may be of a color that is readily visible to the unaided human eye and different than the color of the first image element. In one embodiment, the second image element is blue.

As the needle is advanced towards the target the third image element 250 may be projected directly onto the patient adjacent to the entry point. In some embodiments, the third image element 250 may be an alphanumeric representation of the distance to the target. Where the alphanumeric representation is a number, it will be negative if the clinician/user overshoots the target. In addition, when instrument 117 advances to within a certain range of the target, a fourth image element 255 representative of a distance to target is projected onto the patient in the form of a dynamic closed geometric shape that surrounds instrument 117's entry point and contracts as instrument 117 moves closer to the target. In some embodiments, the fourth image element 255 is a circle. In some embodiments, the third and fourth image elements 250, 255 may be of a color that is readily visible to the unaided human eye and different than the first and second image elements 240, 245. In one embodiment, the third and fourth image elements 250, 255 are green.

An example of an AR/MRI-guided needle intervention in accordance with an embodiment of the invention is described. Multi-modal fiducial markers 125 are placed around the intervention area on the patient. The area of interest is then MRI scanned. In this embodiment, multi-modal markers 125 must be visible under MRI to permit control unit 200 to segment them in the MRI volume. Control unit 200 registers MRI volume to camera view automatically as soon as the markers come into the field of view. A needle trajectory may be selected by a clinician/user, by for example, identifying and contacting an entry point on volumetric image 215 on the touch screen display of control unit 200 and contacting a target point on volumetric image 215 on the touch screen display of control unit 200. Without further user intervention, the guidance indicators (guidance feedback image) are projected on the patient helping the physician place the needle tip at the entry point and aligning the needle with the planned trajectory. The projected guidance indicators also show the progress of the needle towards the target. A live augmented camera view and cross-sections of the MRI volume with guidance feedback images may be shown on touchscreen 210. In accordance with an aspect of the invention, this procedure may be performed outside the bore thereby giving the clinician/user easy access to the intervention site yet inside. Also, advantageously, the procedure may be performed within the MR suite. This allows imaging and intervention to be conducted in one visit and likely reduces that overall number of MR scans need to successfully complete an intervention.

An example of an AR/CT guided procedure in accordance with an embodiment of the invention is described. Multi-modal markers are placed on the patient around the area of interest before an initial CT scan. Next, a CT scan of the patient is acquired and the CT volume is transmitted to control unit 200. Upon receiving the CT volume, control unit 200 automatically loads the CT volume and segments the multi-modal markers in it. The clinician/user inputs a target and an entry point to control unit 200. Once the target is selected and the patient is brought into the field of view of cameras 125, the markers are registered, and projector 130 projects guidance indicators on the patient without any user input. At this point, the clinician/user can simply place the needle tip on the projected entry point and orient the needle to minimize the projected “needle shadow”. The projected guidance will show how close the needle tip is to the target as the needle approaches the target.

To facilitate operation with both MRI and CT scanners, the present invention employs multi-modal markers 125 that are visible under both MRI and CT. Preferred multi-modal markers include those developed and sold by Clear Guide Medical under the VISIMARKER® trademark.

The system may be configured to provide one or more channels for communicating with a clinician or robot using visible light or invisible IR/RF. This could be projected onto the patient or directly radiated towards another device.

The system described herein may be configured to provide real time feedback interface to guide a procedure using any scan that can detect the multi modal markers.

The system may be configured to detect and adjust guidance and interface for changes in attitude and inclination

The system may be further configured to reconstruct the surface of the patient and then use the reconstructed surface to (i) detect deformation caused by the instrument, (ii) adjust the projected guidance based on the curvature of the surface, and (iii) determine patient motion/breathing motion and both project feedback regarding the motion and also adjust the projected guidance based on that feedback.

The system may be still further configured to exhibit adaptive projection to adjust the location of projection of the guidance indicators, to use the live feed from cameras 125 to adjust the appearance of the guidance indicators to account for skin tone, curvature, etc. This may be accomplished by changing the hue, adjusting the brightness or contrast or size of the guidance indicators.

The system may be configured to highlight or project onto the instrument itself as a way of providing feedback to the clinician/user and also to aid the accurate detection of the instrument.

The system may be configured to project where to put the instrument on the patient and how to orient it (not just the needle but also other imaging devices/instruments such as an ultrasound probe).

The foregoing detailed description of embodiments includes references to the drawings or figures, which show illustrations in accordance with example embodiments. The embodiments described herein can be combined, other embodiments can be utilized, or structural, logical and operational changes can be made without departing from the scope of what is claimed. The foregoing detailed description is, therefore, not to be taken in a limiting sense, and the scope is defined by the appended claims and their equivalents. It should be evident that various modifications and changes can be made to these embodiments without departing from the broader spirit and scope of the present disclosure. Accordingly, the specification and drawings are to be regarded in an illustrative rather than a restrictive sense.

Present teachings may be implemented using a variety of technologies. For example, certain aspects of this disclosure may be implemented using electronic hardware, computer software, or any combination thereof. Whether such elements are implemented as hardware or software depends upon the particular application and design constraints imposed on the overall system. By way of example, control unit 200 may be implemented with a processing system that includes one or more processors. Examples of processors include microprocessors, microcontrollers, Central Processing Units (CPUs), digital signal processors (DSPs), field programmable gate arrays (FPGAs), programmable logic devices (PLDs), state machines, gated logic, discrete hardware circuits, and other suitable hardware configured to perform various functions described throughout this disclosure. One or more processors in the processing system may execute software, firmware, or middleware (collectively referred to as “software”). The term “software” shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise. In certain embodiments, the electronic hardware can also include designed application-specific integrated circuits (ASICs), programmable logic devices, or various combinations thereof. The processing system can refer to a computer (e.g., a desktop computer, tablet computer, laptop computer), cellular phone, smart phone, and so forth. The processing system can also include one or more input devices, one or more output devices (e.g., a display), memory, network interface, and so forth.

If certain functions described herein are implemented in software, the functions may be stored on or encoded as one or more instructions or code on a non-transitory computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), compact disk ROM (CD-ROM) or other optical disk storage, magnetic disk storage, solid state memory, or any other data storage devices, combinations of the aforementioned types of computer-readable media, or any other medium that can be used to store computer executable code in the form of instructions or data structures that can be accessed by a computer.

For purposes of this disclosure, the term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

1. A guidance system for targeted interventions using medical instruments comprising:
an optical unit having first and second cameras and a projector disposed between the first and second cameras, said optical head being positioned such that a desired area of intervention defined by plurality of multi modal markers is within a field of view;
a control unit having a touch screen display, said control unit communicatively coupled with said optical unit and configured to receive a real time image of the area of intervention, said control unit being configured to (i) receive MRI volume representations of the desired area of intervention, (ii) register multi modal markers that appear in the field of view, (iii) generate guidance feedback images comprising a plurality of image elements based on the MRI volume representations and the real time image responsive to multi modal marker registration and (iii) to transmit the guidance feedback images to the projector;
the projector directing at least one of the guidance feedback image elements onto the area of intervention responsive to receipt of the guidance feedback images from said control unit.

2. The guidance system of claim 1 wherein said control unit displays a desired instrument entry point and a target on the MRI volume representation of the intervention area responsive to user selection.

3. The guidance system of claim 2 wherein the guidance feedback image includes a first image element representing a point of entry for the medical instrument, a second image element representing an orientation of the medical instrument and a third image element representing a distance to the target.

4. The guidance system of claim 3 wherein the projector directs the first image element onto the area of intervention.

5. The guidance system of claim 3 wherein the projector directs the first image element onto the area of intervention and responsive to the intervention area being contacted by a medical instrument, directs the second image element onto the area of intervention.

6. The guidance system of claim 3 wherein the first image element is a small circle, the second image element is a shadow line and the third image element is an alpha numeric representation of a distance to the target and as the medical instrument is aligned with the shadow line, the shadow line is minimized.

7. The guidance system of claim 6 wherein as the medical instrument is inserted into the area of intervention through the circle and approaches the target, the circle begins to close about the medical instrument.

8. The guidance system of claim 6 wherein the projector directs the third image element onto the area of intervention which is updated in real time as the medical instrument approaches the target.

9. The guidance system of claim of claim 1 wherein one or more of the guidance feedback image elements are displayed on the touch screen.

10. The guidance system of claim 9 wherein the real time image of the area of intervention is displayed on the touch screen.

11. The guidance system of claim 1 further comprising a mounting arm, said optical head being rotatably mounted to said mounting arm.

12. The guidance system of any of claim 3 wherein said guidance system components are MRI safe.

13. A method of guiding an instrument to a target for guided intervention:
displaying a volumetric image of an intervention area on a touch screen display;
generating an instrument path over the volumetric image responsive to input to a user contacting the touch screen display at selected points of the volumetric image;
generating a guidance feedback image responsive to instrument contact with the intervention area of the subject, the guidance feedback image including a plurality of image elements;
projecting a first image element onto the subject, the first image element indicating any entry point for a medical instrument;
projecting a second image element onto the subject, the second image element indicating a trajectory guide for the medical instrument, the second image element comprising a dynamic shadow line;
contracting the shadow line to a point when the instrument is aligned with the instrument path; and
projecting a third image element onto the subject, the third image element indicating a distance from a tip of the medical instrument to the target.

14. The method of claim 13 wherein said first image element is a closed geometric shape.

15. The method of claim 14 the first image element is a circle of a first color that is visible to the unaided human eye.

16. The method of claim 13 wherein the second image element is a dynamic line of a second color that is different from the first color and is readily visible to the unaided human eye.

17. The method of claim 13 wherein the third image element is an alphanumeric indication of the distance from a tip of the medical instrument to the target.

18. The method of claim 13 further comprising projecting a fourth image element onto the subject, the fourth image element is a dynamic closed geometric shape that contracts as the tip of the medical instrument approaches the target.
